(19) United States
(12) Patent Application Publication
(10) Pub. No.: US 2007/0100252 A1
(43) Pub. Date: May 3, 2007

(54) PORTABLE ELECTRONIC DEVICE WITH MEASURING FAT FUNCTION AND MEASURING FAT METHOD THEREOF

(75) Inventors: Chung-Cheng Chou, Taoyuan County (TW); Po-Son Wang, Tainan County (TW)

Correspondence Address: THOMAS, KAYDEN, HORSTEMEYER & RISLEY, LLP, 100 GALLERIA PARKWAY, NW, STE 1750, ATLANTA, GA, 30339-5948, US

(73) Assignee: BENQ CORPORATION, Taoyuan Shien (TW)

(21) Appl. No.: 11/538,822

(22) Filed: Oct. 5, 2006

(30) Foreign Application Priority Data
Oct. 20, 2005 (TW) 94136726

(51) Int. Cl. A61B 5/05

(52) U.S. Cl. 600/547

(57) ABSTRACT

A portable electronic device with fat measuring function and a fat measuring method thereof are provided. The portable electronic device includes a first button, a second button and a fat-measuring controlling circuit. The first button and the second button are made from a conductive material. The fat-measuring controlling circuit is coupled with the first button and the second button respectively. When the user touches the first button and the second button, the fat-measuring controlling circuit measures an electrical signal between the first button and the second button to generate the user's body fat rate.

This application claims the benefit of Taiwan application Serial No. 94136726, filed Oct. 20, 2005, the subject matter of which is incorporated herein by reference.

### BACKGROUND OF THE INVENTION

1. Field of the Invention

The invention relates in general to a portable electronic device, and more particularly to a portable electronic device with fat measuring function and fat measuring method thereof.

2. Description of the Related Art

Bioelectric impedance analysis (BIA) is a technology for measuring the composition of the body. Different parts of the body have different conductivities. When a micro-current (approximately ranges from 200 μA to 800 μA) whose frequency is over 50 KHz is applied to a tissue of the body, the current will flow through the liquid outside the cells. Part of the current will be impeded by the cell membranes and generate a resistance R, and part of the current will reduce its speed due to the charge of the cell membranes and generate a reactance I. The sum of the resistance R and the reactance I is the biological impedance Z, and normally the resistance R amounts to more than 90% of the biological impedance Z.

The body fat is not conductive but the water inside the body is conductive. Therefore, the body fat can be estimated according to the measure of the user's biological impedance.

Referring to FIG. 1, a mobile phone with fat measuring function disclosed in U.S. Pat. No. 6,327,495 is shown. The conventional mobile phone includes an sensing electrode A, an sensing electrode B, an sensing electrode C, an sensing electrode D, a function key 9, a display key 10 and a screen 3.

When the user presses the function key 9, the mobile phone shifts to a fat measuring mode from a communication mode. Under the fat measuring mode, the user needs to touch the sensing electrode A, the sensing electrode B, the sensing electrode C and the sensing electrode D at the same time. The sensing electrode A, the sensing electrode B, the sensing electrode C and the sensing electrode D are respectively disposed at the four corners of the mobile phone. When the user touches the abovementioned sensing electrodes, the mobile phone provides a certain volume of current, which flows through the user's body through the abovementioned sensing electrodes to generate a biological impedance Z. The user's body fat can be calculated according to the abovementioned biological impedance Z. Lastly when the user presses the display key 10, the screen 3 displays the measuring results.

The sensing electrode A, the sensing electrode B, the sensing electrode C and the sensing electrode D are respectively disposed at the four corners of the mobile phone. When the user would like to measure the body fat, the user has to touch the sensing electrodes disposed at the four corners of the mobile phone at the same time. Such design is user unfriendly and having too many buttons not only adds more difficulties to the user but also incurs more costs. Moreover, the conventional mobile phone can measure the body fat only when the mobile phone is turned on and can not measure the body fat when the mobile phone is turned off.

### SUMMARY OF THE INVENTION

It is therefore an object of the invention to provide a portable electronic device with fat measuring function. The portable electronic device is incorporated with the sensing electrode for measuring body fat rate, hence enabling modern people who are busy with their work to conveniently measure their body fat for health management.

The invention achieves a first object by providing a portable electronic device with fat measuring function. The portable electronic device includes a first button, a second button and a fat-measuring controlling circuit. The first button and the second button are made from a conductive material. The fat-measuring controlling circuit is coupled with the first button and the second button respectively. When the user touches the first button and the second button, the fat-measuring controlling circuit measures an electrical signal between the first button and the second button to generate the user's body fat rate.

The invention achieves a second object by providing a fat measuring method of a portable electronic device. The fat measuring method includes the following steps. Firstly, a function key of the portable electronic device is activated. Next, a first button and a second button of the portable electronic device are set as a first sensing electrode and a second sensing electrode respectively. The first button and the second button are made from a conductive material. Then, when the first sensing electrode and the second sensing electrode are touched by the user, an electrical signal between the first button and the second button is measured. Lastly, the user's body fat rate is generated according to the electrical signal.

The invention further achieves a third object by providing a portable electronic device with fat measuring function. The portable electronic device includes a first sensing electrode, a second sensing electrode, a flip cover, an electronic device body and a fat-measuring controlling circuit. The flip cover is pivotally connected to one end of the electronic device body. The first sensing electrode is disposed on the surface of the flip cover. The second sensing electrode is disposed on the surface of the electronic device body. The fat-measuring controlling circuit is coupled with the first sensing electrode and the second sensing electrode respectively. When the user flips the flip cover and touches both the first sensing electrode and the second sensing electrode, the fat-measuring controlling circuit measures an electrical signal between the first sensing electrode and the second sensing electrode to generate the user's body fat rate.

The invention further achieves a fourth object by providing a portable electronic device with fat measuring function. The portable electronic device includes an electronic device body, a first sensing electrode, a second sensing electrode, a magnetism-electricity converter and a fat-measuring controlling circuit. The first sensing electrode and the second sensing electrode are removably disposed on the electronic device body, The magnetism-electricity converter is electrically connected to the first sensing electrode and the second sensing electrode. When the user enables the first sensing electrode and the second sensing electrode to move relatively to the electronic device body, the magnetism-electricity converter correspondingly outputs a first current. The fat-measuring controlling circuit receives the first current and outputs a second current to the first sensing electrode, and then measures an electrical signal between the first sensing electrode and the second sensing electrode to generate the user's body fat rate.

Other objects, features, and advantages of the invention will become apparent from the following detailed description of the preferred but non-limiting embodiments. The following description is made with reference to the accompanying drawings.

### BRIEF DESCRIPTION OF THE DRAWINGS

FIG. 1 is a mobile phone with fat measuring function disclosed in U.S. Pat. No. 6,327,495;

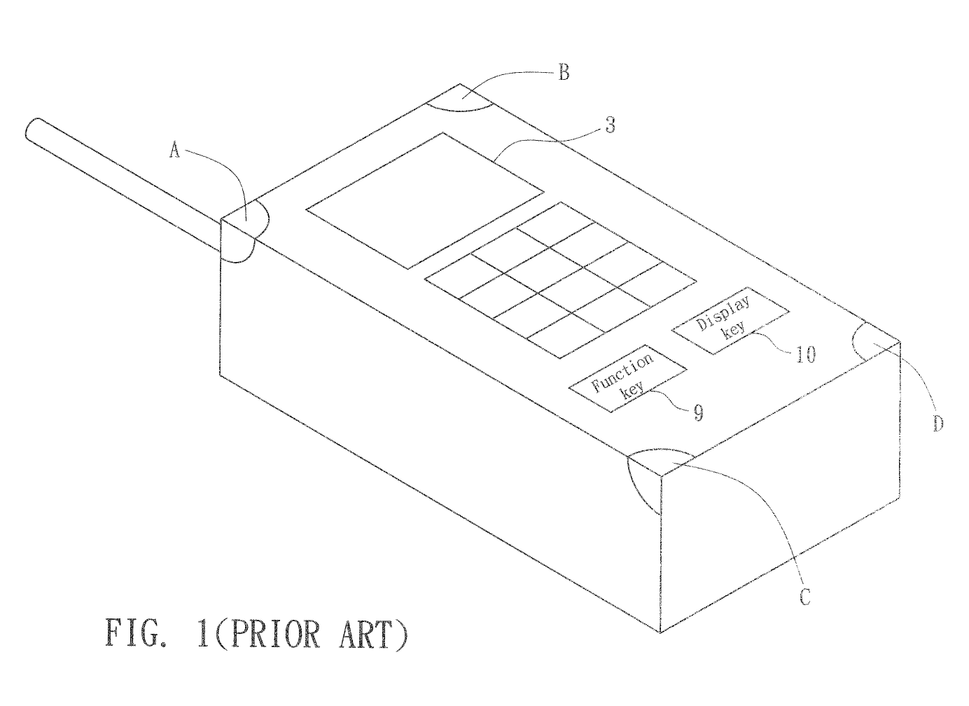

FIG. 2 is a front view of a portable electronic device with fat measuring function according to a first embodiment of the invention;

FIG. 3 is a partial block diagram of a portable electronic device with fat measuring function according to the first embodiment of the invention;

FIG. 4 is a flowchart for a fat measuring method of a portable electronic device;

FIG. 5 and FIG. 6 are two side views of a portable electronic device with fat measuring function according to a second embodiment of the invention;

FIG. 7 is a front view of a portable electronic device with fat measuring function according to a third embodiment of the invention;

FIG. 8 is a partial block diagram of a portable electronic device with fat measuring function according to the third embodiment of the invention;

FIG. 9 is a detailed circuit diagram of FIG. 8;

FIG. 10 is a 3-dimensional diagram of a rotation mechanism; and

FIG. 11 is a top view of the rotation mechanism.

### DETAILED DESCRIPTION OF THE INVENTION

### First Embodiment

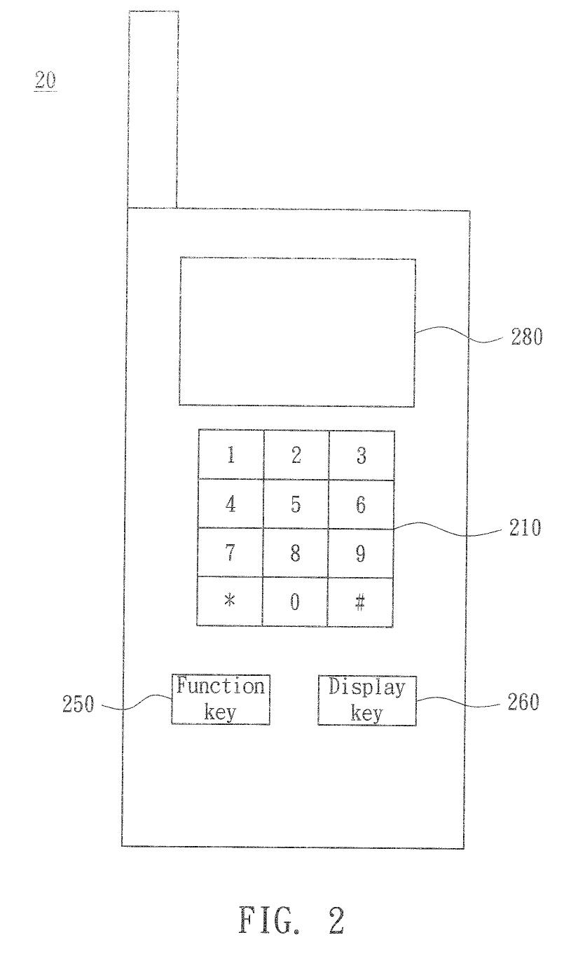

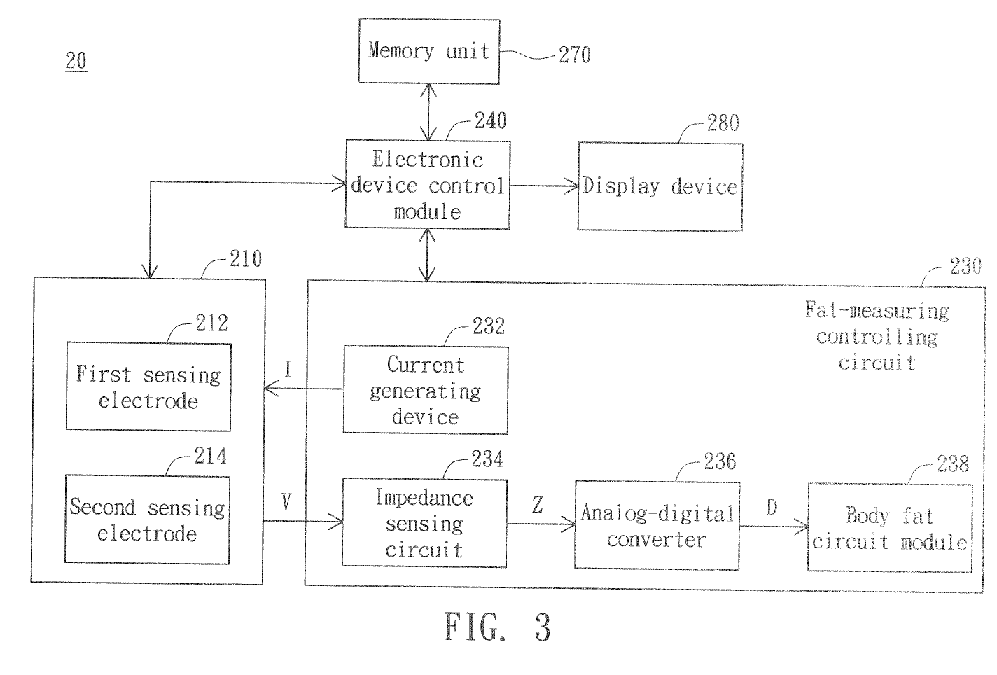

Referring to both FIG. 2 and FIG. 3. FIG. 2 is a front view of a portable electronic device with fat measuring function according to a first embodiment of the invention. FIG. 3 is a partial block diagram of a portable electronic device with fat measuring function according to the first embodiment of the invention. The portable electronic device 20 includes a alpha-numeric key pad 210, a fat-measuring controlling circuit 230, an electronic device control module 240, a function key 250, a display key 260, a memory unit 270 and a display device 280.

The alpha-numeric key pad 210 of the portable electronic device 20 is made from a highly conductive resin material. The highly conductive resin material comprises acetal resin, carbon black composites, epoxide composites and polyester copolymer The fat-measuring controlling circuit 230 is coupled with the key pad 210. The key pad 210, the fat-measuring controlling circuit 230, the memory unit 270 and the display device 280 are respectively coupled with the electronic device control module 240.

The function key 250 of the portable electronic device 20 is for changing the operation mode of the key pad 210. For example, if the portable electronic device is a mobile phone, before the function key 250 is activated, the key pad 210 can be used for dialing a phone number or writing a text message. When function key 250 is activated, the user can set any two buttons of the key pad 210 as the first sensing electrode 212 and the second sensing electrode 214 for measuring the body fat rate.

Afterwards, when the first sensing electrode 212 and the second sensing electrode 214 are touched by the user, the fat-measuring controlling circuit 230 measures an electrical signal between the first sensing electrode 212 and the second sensing electrode 214. Examples of the electrical signal include a voltage difference. The portable electronic device 20 generates a body fat rate for the user according to the electrical signal. The portable electronic device 20 stores the user's body fat rate in the memory unit 270, and when the user activates the display key 260, the user's body fat rate is displayed on the display device 280.

Furthermore, the fat measuring controlling circuit 230 includes a current generating device 232, an impedance sensing circuit 234, an analog-digital converter 236 and a body fat circuit module 238. The current generating device 232 provides a current I. Example of the current I is a micro current ranging from 200 μA to 800 μA whose frequency is over 50 KHz. The current I flows through the user's body via the first sensing electrode 212 and the second sensing electrode 214 and forms a voltage difference V between the first sensing electrode 212 and the second sensing electrode 214. The impedance sensing circuit 234 detects the voltage difference V and then generates a biological impedance Z according to the voltage difference V. The analog-digital converter 236 generates a digital data D according to the level of the biological impedance Z. The body fat circuit module 238 generates the user's body fat rate according to the digital data D.

Besides, whether the body fat rate is normal depends on the user's age, height, weight and gender. The data of the user's age, height, weight and gender is stored in the memory unit 270 for the portable electronic device 20 to perform further analysis, determine the state of the user's health, and provide relevant messages or suggestions to the user via the display device 280. For example, the user may receive an over-weight warning, a recommended diet or time schedule.

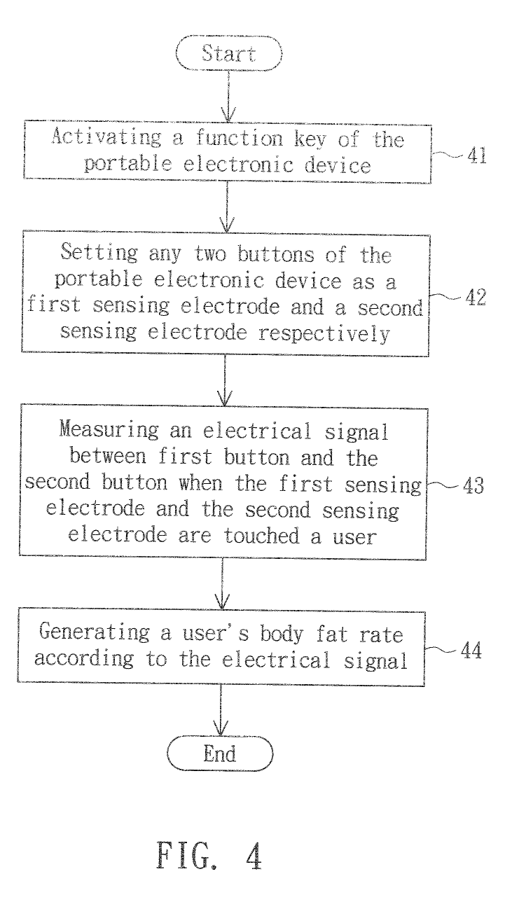

Referring to FIG. 4, a flowchart for a fat measuring method of a portable electronic device is shown. The fat measuring method of the portable electronic device 20 includes the following steps. Firstly, the method begins at step 41, the function key 250 of the portable electronic device 20 is activated. Next, proceed to step 42, any two buttons of the portable electronic device 20 are set as the first sensing electrode 212 and the second sensing electrode 214 respectively. Then, proceed to step 43, when the first sensing electrode 212 and the second sensing electrode 214 are touched by the user, an electrical signal between the two buttons is measured. Lastly, proceed to step 44, a body fat rate is generated according to the electrical signal.

According to the first embodiment, the sensing electrode for measuring the body fat rate is incorporated into the existing buttons of the portable electronic device, hence making the measuring of body fat more convenient.

### Second Embodiment

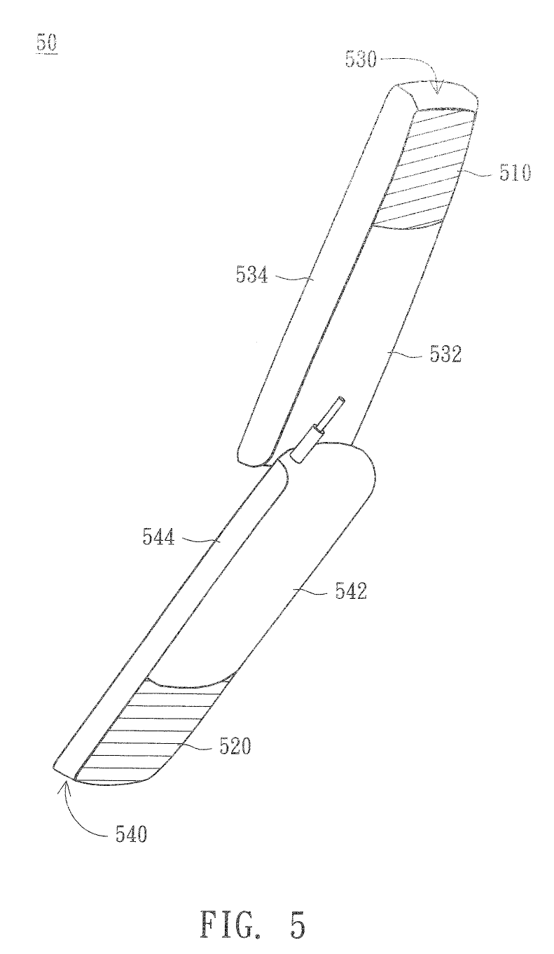

The first sensing electrode and the second sensing electrode can be designed to dispose at a part of the portable electronic device most often touched by the user. Referring to both FIG. 5 and FIG. 6, two side views of a portable electronic device with fat measuring function according to a second embodiment of the invention are shown. The portable electronic device 50 includes a first sensing electrode 510, a second sensing electrode 520, a flip cover 530, an electronic device body 540 and a fat-measuring controlling circuit.

The flip cover 530 of the portable electronic device 50 is pivotally connected to one end of the electronic device body 540. The flip cover 530 has a top surface 532 and a bottom surface 536, and the top surface 532 is opposite to the bottom surface 536. Similarly, the electronic device body 540 has a top surface 546 and a bottom surface 542, and the top surface 546 is opposite to the bottom surface 542. When the flip cover 530 is not flipped, the bottom surface 536 of the flip cover 530 touches the top surface 546 of the electronic device body 540.

The first sensing electrode 510 is disposed on the top surface 532 of the flip cover 530. The second sensing electrode 520 is disposed on the bottom surface 542 of the electronic device body 540. The first sensing electrode 510 and the second sending electrode 520 are preferably disposed at a part of the portable electronic device 50 most often touched by the user when flipping the flip cover 530. For example the first sensing electrode 510 and the second sensing electrode 520 may be disposed at the front end of the flip cover 530 and the bottom end of the electronic device body 540 respectively.

The fat-measuring controlling circuit used in the present embodiment of the invention is similar to the fat-measuring controlling circuit 230 of FIG. 3 and is not repeated here. The fat-measuring controlling circuit is disposed in the electronic device body 540 and is coupled with the first sensing electrode 510 and the second sensing electrode 520 respectively.

When the user flips the flip cover 530 and touches the first sensing electrode 510 and the second sensing electrode 520 at the same time, the fat-measuring controlling circuit measures an electrical signal between the first sensing electrode 510 and the second sensing electrode 520. Example of the electrical signal is a voltage difference. Then, the portable electronic device 50 generates a body fat rate for the user according to electrical signal.

Besides, the disposition of the first sensing electrode 510 and the disposition of the second sensing electrode 520 are not limited to the top surface 532 of the flip cover 530 and the bottom surface 542 of the electronic device body 540. For example, the first sensing electrode 510 and the second sensing electrode 520 can be respectively disposed on a lateral surface 534 of the flip cover 530 and a lateral surface 544 of the electronic device body 540 to measure the user's body fat rate.

In the second embodiment of the invention, the sensing electrode for measuring the body fat rate is disposed at a part most often touched by the user, such that the user is enabled to measure their body fat rate for health management when operating a portable electronic device.

### Third Embodiment

The first sensing electrode and the second sensing electrode can be designed as removable sensing electrodes. When the first sensing electrode and the second sensing electrode are removed from the portable electronic device, the first sensing electrode and the second sensing electrode generate the needed power for measuring the body fat rate without consuming the power of the batteries of the portable electronic device. Therefore, the portable electronic device can measure the user's body fat rate even at a turn-off mode.

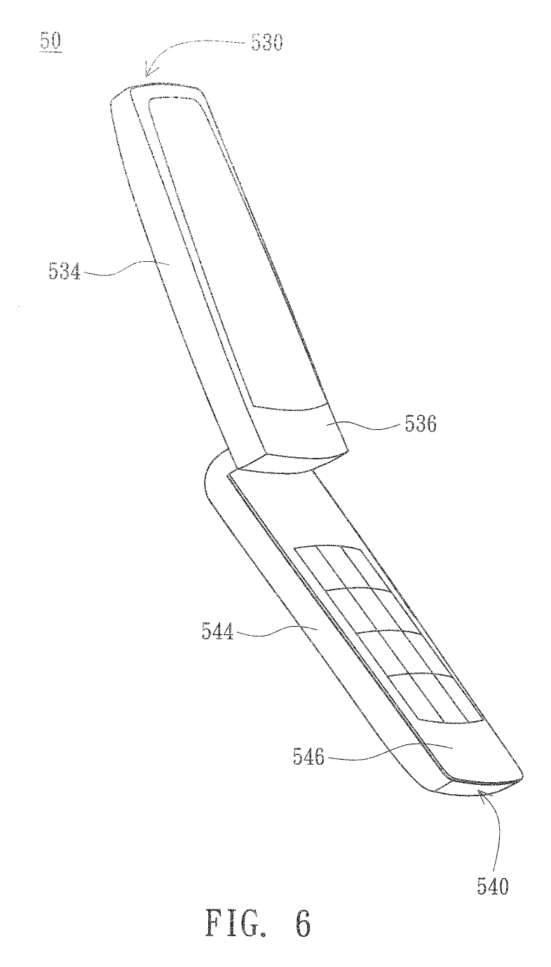

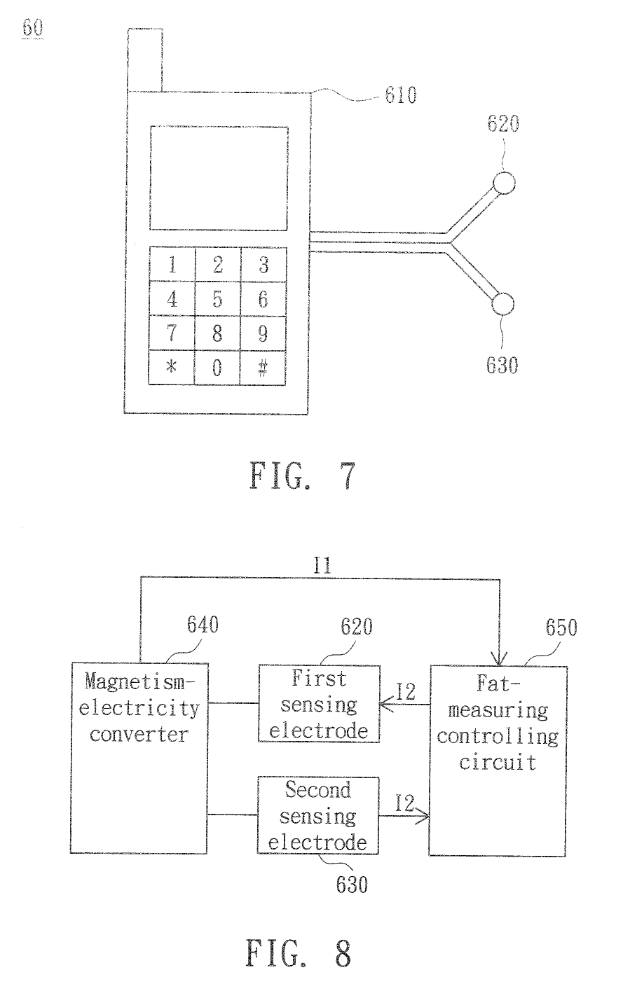

Referring to FIG. 7 and FIG. 8. FIG. 7 is a front view of a portable electronic device with fat measuring function according to a third embodiment of the invention. FIG. 8 is a partial block diagram of a portable electronic device with fat measuring function according to the third embodiment of the invention. The portable electronic device 60 includes an electronic device body 610, a first sensing electrode 620, a second sensing electrode 630, a magnetism-electricity converter 640 and a fat-measuring controlling circuit 650.

In the portable electronic device 60, the first sensing electrode 620 and the second sensing electrode 630 are removably disposed on the electronic device body 610. The magnetism-electricity converter 640 is coupled with the first sensing electrode 620 and the second sensing electrode 630. When the user enables the first sensing electrode 620 and the second sensing electrode 630 to generate relative movement with respect to the electronic device body 610, for example, the first sensing electrode 620 and the second sensing electrode 630 are removed outwardly, the magnetism-electricity converter 640 correspondingly outputs a current I1.

The fat-measuring controlling circuit 650 receives and converts the current I1 into a current I2 and then outputs the current I2 to the first sensing electrode 620. When the user removes the first sensing electrode 620 and the second sensing electrode 630, the user touches the first sensing electrode 620 and the second sensing electrode 630 respectively. Therefore, the current I2 flows through the user's body via the first sensing electrode 620 and the second sensing electrode 630 and forms a voltage difference between the first sensing electrode 620 and the second sensing electrode 630. The fat-measuring controlling circuit 650 measures the voltage difference to generate a user's body fat rate.

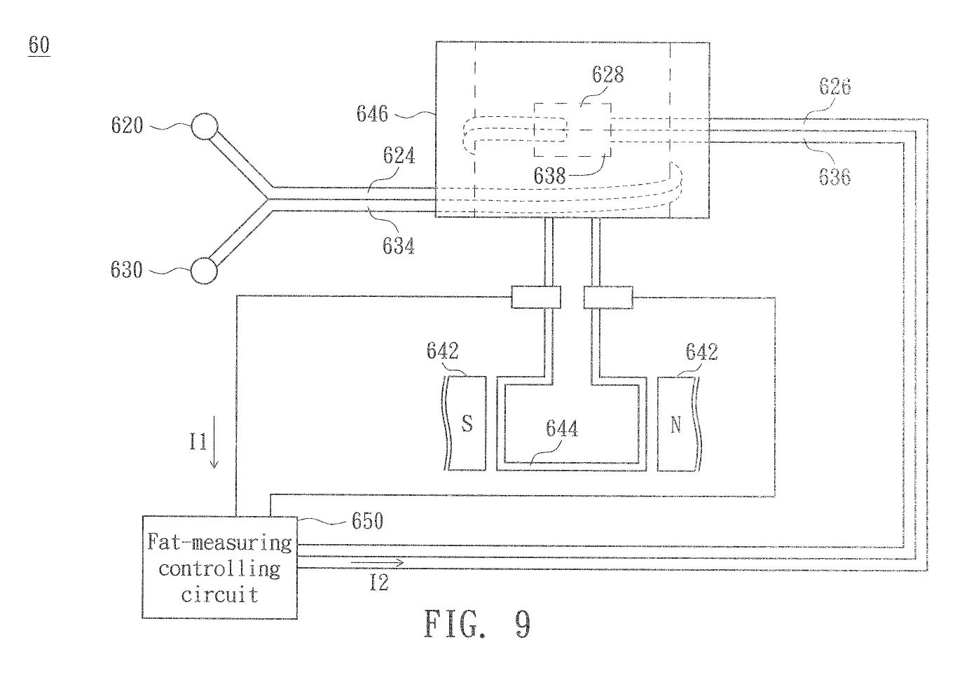

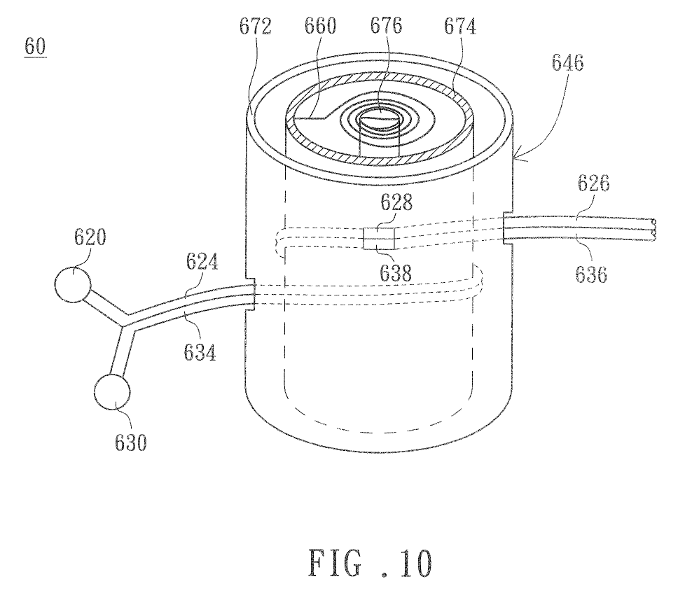

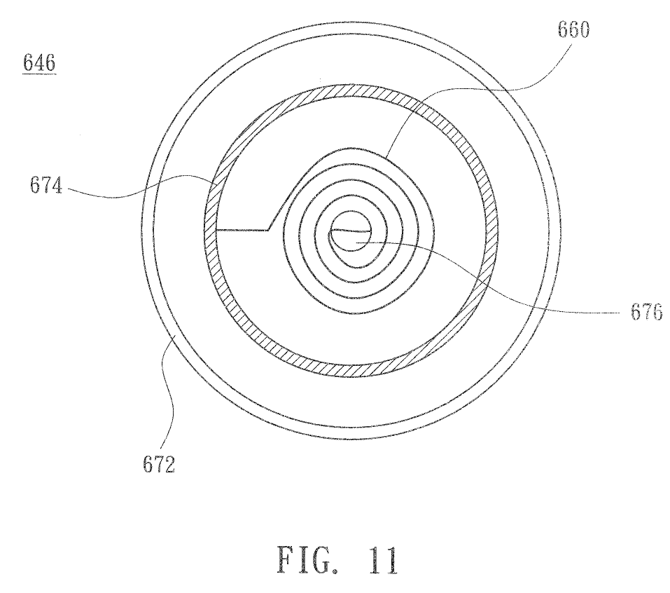

Referring to FIG. 9, FIG. 10 and FIG. 11 at the same time. FIG. 9 is a detailed circuit diagram of FIG. 8. FIG. 10 is a 3-dimensional diagram of a rotation mechanism. FIG. 11 is a top view of the rotation mechanism. Furthermore, the magnetism-electricity converter 640 includes a magnetic field generator 642, an inductance coil 644, a rotation mechanism 646, an electrical connection portion 628 and an electrical connection portion 638. The magnetic field generator 642 provides a magnetic field, such magnetic field is induced by an N-polarity end of magnet and an S-polarity end of magnet as shown in FIG. 9. The inductance coil 644 is disposed in the magnetic field.

The rotation mechanism 646 includes a spring 660, an electrical connection portion 628, an electrical connection portion 638, a housing 672, a fixing pivot 676 and a rotating portion 674. The spring 660 is coupled with the fixing pivot 676 and the rotating portion 674 respectively. The spring 660, the fixing pivot 676 and the rotating portion 674 are disposed in the housing 672.

The electrical connection portion 628 and the electrical connection portion 638 are disposed on the rotating portion 674. One end of the conducting wire 624 is coupled with the first sensing electrode 620, and the other end is fixed on the electrical connection portion 628. One end of the conducting wire 634 is coupled with the second sensing electrode 630, while the other end is fixed on the electrical connection portion 638. Besides, one end of the conducting wire 626 is fixed on the electrical connection portion 628, while the other end is electrically connected to the fat-measuring controlling circuit 650. One end of the conducting wire 636 is fixed on the electrical connection portion 638, while the other end is electrically connected to the fat-measuring controlling circuit 650.

The conducting wire 624 and the conducting wire 634 are coiled around the rotating portion 674. When the first sensing electrode 620 and the second sensing electrode 630 are pulled, the conducting wire 624 and the conducting wire 634 drive the rotating portion 674 to rotate between the housing 672 and the fixing pivot 676. The rotating portion 674 drives the inductance coil 644 to rotate, and further cuts the magnetic field to correspondingly output the current I1.

The current I1 is inputted to the fat-measuring controlling circuit 650 so that the fat-measuring controlling circuit 650 is provided with necessary power for normal operation. When measuring the body fat rate, the fat-measuring controlling circuit 650 outputs a current I2. The current I2 is inputted to the electrical connection portion 628 via the conducting wire 626 and flows to the user via the conducting wire 624 and the first sensing electrode 620. After the current I2 flows through the user, the current I2 flows back to the fat-measuring controlling circuit 650 via the second sensing electrode 630, the conducting wire 634, the electrical connection portion 638 and the conducting wire 636. After the current I2 flows through the user, a voltage difference is formed between the first sensing electrode 620 and the second sensing electrode 630. The fat-measuring controlling circuit 650 estimates a body fat rate for the user according to the voltage difference.

The spring 660 is coupled with the rotating portion 674 and the fixing pivot 676 respectively. Therefore, when the measuring is finished and the user releases the first sensing electrode 620 and the second sensing electrode 630, the spring 660 generates an elasticity for enabling the rotating portion 674 to rotate with respect to the fixing pivot 676, so that the first sensing electrode 620 and the second sensing electrode 630 are pushed back to their original position.

In the third embodiment, the sensing electrodes for measuring body fat rate are designed as removable sensing electrodes. When the user is ready to measure the body fat rate, the needed power is generated when the sensing electrode is removed, so that the portable electronic device can generate the needed current for measuring the body fat rate without consuming the power of the batteries. Therefore, the portable electronic device still can measure the body fat rate even at a turn-off mode.

Examples of the portable electronic device disclosed in the first embodiment, the second embodiment and the third embodiment include a mobile phone, a personal digital assistant, an MP3 player, a game station, a watch, a walkman, a digital camera or a handy drive. The sensing electrode is skillfully incorporated in the portable electronic device to make the measurement of body fat rate much more convenient.

According to the portable electronic device with fat measuring function disclosed in the above embodiments of the invention, the sensing electrode for measuring body fat rate is incorporated in a portable electronic device, hence enabling the user to conveniently measure their body fat for health management.

While the invention has been described by way of example and in terms of a preferred embodiment, it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A portable electronic device, comprising:
a key pad allowing a user input a alpha-numeric data, the key pad comprising a firs button and a second button, both the first button and the second button being made from a conductive material, and
a fat-measuring controlling circuit coupled with the first button and the second button respectively;
wherein when a user touches the first button and the second button, the fat-measuring controlling circuit measures an electrical signal between the first button and the second button to generate the user's a body fat rate.

2. The portable electronic device according to claim 1, wherein the first button and the second button are made from a highly conductive resin material.

3. The portable electronic device according to claim 2, wherein the highly conductive resin material for making the first button and the second button comprises acetal resin, carbon black composites, epoxide composites and polyester copolymer.

4. The portable electronic device according to claim 2, wherein the portable electronic device comprises:
an electronic device control module coupled with the first button and the second button;
a function key, wherein after the function key is activated, the first button is set as a first sensing electrode and the second button is set as a second sensing electrode, and when the first sensing electrode and the second sensing electrode are both touched by the user, the portable electronic device generates the user's body fat rate accordingly.

5. The portable electronic device according to claim 4, wherein portable electronic device comprises:
a display device coupled with the electronic device control module to display the body fat rate; and
a memory unit coupled with the electronic device control module to store the body fat rate.

6. The portable electronic device according to claim 5, wherein the electronic device further comprises a display key, and when the display key is activated, the body fat rate is displayed on the display device.

7. The portable electronic device according to claim 4, wherein the fat-measuring controlling circuit comprises:
a current generating device for providing a current;
an impedance sensing circuit, wherein when the user touches the first sensing electrode and the second sensing electrode, the current flows through the user to form a voltage difference correspondingly between the first sensing electrode and the second sensing electrode, the impedance sensing circuit detects the voltage difference between the first sensing electrode and the second sensing electrode to generate an impedance according to the voltage difference;
an analog-digital converter for receiving the impedance to generate a digital data according to the impedance; and
a body fat circuit module for generating a body fat rate according to the digital data.

8. The portable electronic device according to claim 1, wherein the portable electronic device is a mobile phone, a personal digital assistant (PDA), an MP3 player, a game station, a watch, a walkman, a digital camera or a handy drive.

9. A fat measuring method of a portable electronic device, the method comprising:
activating a function key of the portable electronic device;
setting a first button and a second button of the portable electronic device as a first sensing electrode and a second sensing electrode respectively, the first button and the second button being made from a conductive material;
measuring an electrical signal between first button and the second button when the first sensing electrode and the second sensing electrode are touched by a user; and
generating a user's body fat rate according to the electrical signal.

10. The fat measuring method according to claim 9, wherein the first button and the second button are made from a highly conductive resin material.

11. The fat measuring method according to claim 10, wherein the highly conductive resin material for the first button and the second button comprises acetal resin, carbon black composites, epoxide composites and polyester copolymer.

12. A port able electronic device, comprising:
a first sensing electrode and a second sensing electrode;
a flip cover, the first sensing electrode being disposed on one of the surfaces of the flip cover; and
an electronic device body, the flip cover being pivotally connected to the one end of the electronic device body, and the second sensing electrode being disposed on one of the surfaces of the electronic device body; and
a fat-measuring controlling circuit coupled with the first sensing electrode and the second sensing electrode;
wherein when the user flips the flip cover and touches the first sensing electrode and the second sensing electrode at the same time, the fat-measuring controlling circuit measures an electrical signal between the first sensing electrode and the second sensing electrode to generate a body fat rate for the user.

13. The portable electronic device according to claim 12, wherein the flip cover has a first top surface and a first bottom surface, the first top surface is opposite to the first bottom surface, the electronic device body has a second top surface and a second bottom surface, the second top surface is opposite to the second bottom surface, the first bottom surface touches the second top surface when the flip cover is not flipped, and the first sensing electrode is disposed on the first top surface.

14. The portable electronic device according to claim 12, wherein the flip cover has a first top surface and a first bottom surface, the first top surface is opposite to the first bottom surface, the electronic device body has a second top surface and a second bottom surface, the second top surface is opposite to the second bottom surface, the first bottom surface touches the second top surface when the flip cover is not flipped, and the second sensing electrode is disposed on the second bottom surface.

15. The portable electronic device according to claim 12, wherein one of the first sensing electrode and the second sensing electrode is disposed on a lateral surface of the flip cover.

16. The portable electronic device according to claim 12, wherein the portable electronic device is a mobile phone, a personal digital assistant, an MP3 player, a game station, a watch, a walkman, a digital camera or a handy drive.

17. A portable electronic device, comprising:
an electronic device body;
a first sensing electrode and a second sensing electrode removably disposed on the electronic device body;
a magnetism-electricity converter coupled with the first sensing electrode and the second sensing electrode, wherein when a user enables the first sensing electrode and the second sensing electrode to move relatively to the electronic device body, the magnetism-electricity converter correspondingly outputs a first current; and
a fat-measuring controlling circuit for receiving the first current and outputting a second current to the first sensing electrode and measuring an electrical signal between the first sensing electrode and the second sensing electrode to generate a body fat rate for the user.

18. The portable electronic device according to claim 17, wherein the magnetism-electricity converter comprises:
a magnetic field generator for providing a magnetic field;
an inductance coil disposed in the magnetic field;
a rotation mechanism, wherein when the user enables the first sensing electrode and the second sensing electrode to move relatively to the mobile phone body, the rotation mechanism drives the inductance coil to rotate so as to cut the magnetic field and correspondingly outputs the first current; and
a first electrical connection portion and a second electrical connection portion both disposed on the rotation mechanism, the fat-measuring controlling circuit being electrically connected to the first sensing electrode via the first electrical connection portion, and the second electrical connection portion being electrically connected to the second sensing electrode to output the second current.

19. The portable electronic device according to claim 18, wherein the portable electronic device further comprises:
a first transmission line, comprising:
a first conducting wire, one end of the first conducting wire being fixed on the first electrical connection portion, and the other end of the first conducting wire being electrically connected to the fat-measuring controlling circuit; and
a second conducting wire, one end of the second conducting wire the being fixed on the second electrical connection portion, and the other end of the second conducting wire being electrically connected to the fat-measuring controlling circuit; and
a second transmission line, comprising:
a third the conducting wire, one end of the third the conducting wire being fixed on the first electrical connection portion, and the other end of the third the conducting wire being coupled with the first sensing electrode; and
a fourth the conducting wire, one end of the fourth the conducting wire being fixed on the second electrical connection portion, and the other end of the fourth the conducting wire being coupled with the second sensing electrode;
wherein the second transmission line is coiled around the rotation mechanism, and when a user pulls the first sensing electrode and the second sensing electrode, the second transmission line drives the rotation mechanism to rotate.

20. The portable electronic device according to claim 17, wherein the portable electronic device is a mobile phone, a personal digital assistant, an MP3 player, a game station, a watch, a walkman, a digital camera or a handy drive.
